(19) United States
(12) Patent Application Publication
(10) Pub. No.: US 2004/0197828 A1
(43) Pub. Date: Oct. 7, 2004

(54) Method for diagnosis and treatment of bone turnover

(76) Inventor: Dana P. Gaddy, Little Rock, AR (US)

Correspondence Address: BUTLER, SNOW, O'MARA, STEVENS & CANNADA PLLC, 6075 POPLAR AVENUE, SUITE 500, MEMPHIS, TN, 38119, US

(21) Appl. No.: 10/810,005

(22) Filed: Mar. 26, 2004

Related U.S. Application Data
(60) Provisional application No. 60/457,710, filed on Mar. 26, 2003.

(51) Int. Cl. G01N 33/53

(52) U.S. Cl. 435/7.1

(57) ABSTRACT

The present invention related to a method for diagnosing, screening, prognosing and treating bone loss as a result of bone turnover in men and women subjects. In particular, bone loss can be predicted in perimenopausal women by measuring Inhibin A levels in serum of a female subject. Similarly, inhibin B can be used to detect idiopathic osteoporosis, in the serum of a male patient.

### FIELD OF THE INVENTION

This invention relates to a method for diagnosing, screening, prognosing and treating disease involving bone loss in humans.

### BACKGROUND OF THE INVENTION

Estrogen plays a critical role in the maintenance of bone homeostasis, and estrogen deficiency in post-menopausal women resulting from depression of both steoblast (OBL) and osteoclast (OCL) development, leading to the loss of bone mass. The pathophysiology of postmenopausal osteoporosis involves an overproduction of osteoclasis, relative to the integrally coupled increase in osteoblastogenesis, a process that, itself, facilitates the support of osteoclast development.

However, recent data have suggested that some clinical indices of increased bone turnover can first be detected in late premenopausal women with normal circulating estrogen levels. Ebeling P R, Atley L M, Guthrie J R, Burger H G, Dennerstein L, Hopper J L, Wark J D. Bone Turnover Markers and Bone Density Across the Menopausal Transition. J Clin. Endocrinol Metab. 81:3366-71 (1996). Thus, this increased bone turnover must be nonsex steroid-dependent. Indeed, the endocrine parameter best correlated with this increase is elevated serum FSH levels. This early rise in FSH levels in perimenopausal women is attributable to a selective decrease in inhibin B secretion. The decrease in inhibin B secretion occurs in the presence of normal levels of E2, inhibin A, GnRH, and L H. Klein N A, Illingworth P J, Groome N P, McNeilly A S, Battaglia D E, Soules M R. Decreased Inhibin B Secretion is Associated with the Monotropic FSH Rise in Older, Ovulatory Women: A Study of Serum and Follicular Fluid Levels of dimeric Inhibin A and B in Spontaneous Menstrual Cycles. J. Clin. Endocrinol Metab. 81:2742-45 (1996). Because both inhibin A and inhibin B isoforms selectively inhibit pituitary FSH secretion, these data suggest that increased FSH is attributable to a loss in feed-back inhibition by gonadal inhibin B in perimenopausal women, resulting in bone loss before the loss of sex steroids. As the loss of gonadal function progresses in postmenopausal women, the well-established decreased in E2 accompany declining levels of both inhibin B and inhibin A, further increasing serum FSH and markedly increasing bone loss.

Inhibin B and inhibin A are heterodimeric proteins in the TGF Beta superfamily composed on αBB subunits, respectively. Inhibins were originally identified based on their ability to suppress pituitary FSH secretion. Vale W, Bilezikjian L M, Rivier C. Reproductive and Other Roles of Inhibins and Activins. In: Knobil E, Neil J D, eds. The physiology of reproduction. New York: Raven Press; 1861-78 (1994). Suppression of FSH by the inhibins is antagonized by the related peptide, activin A, a homodimer composed of BA BA subunits that is locally produced in the gonad. Vale W, Bilezikjian L M, Rivier C. Reproductive and Other Roles of inhibins and Activins. In: Knobil E, Neil J D, eds. The physiology of reproduction. New York: Raven Press; 1861-78 (1994). In addition to opposing effects on pituitary FSH production and gonadal steroid production, inhibins and activin exert opposing effects on erythroid (Yu J, Shao L E, Lemas V, Yu A L, Vaughan J, Rivier J, Vale W. Importance of FSH-Releasing Protein and Inhibin in Erythrodifferentiation. Nature 330:765-767 (1987)), megakaryocyte (Fujimoto K, Kawakita M, Kato K, Yonemura Y, Masuda T, Matsuzaki H, Hirose J, Isaji M, Sasaki H, Inoue T. Purifi cation of Megakaryocyte Differentiation Activity from a Human Fibrous Histiocytoma Cell Line: N-Terminal Sequence Homology with Activin A. Biochem Biophys Res Commun 174:1163-68 (1991)), and granulocyte-macrophage cell development (Broxmeyer H E, Lu L, Cooper S, Schwall R H, Mason A J, Nikolics K. Selective and Indirect Modulation ofHuman Multipotential and Erythroid Hematopoietic Progenitor Cell Proliferation by Recombinant Human Activin and Inhibin. Proc Natl Acad Sci USA 85:9052-56 (1988)). Activin BA subunit mRNA is also locally produced in bone marrow (Yu A W, Shao L E, Frigon Jr N L, Yu J. Detection of Functional and Dimeric Activin A in Human Marrow Microenvironment: Implications for the Modulation of Erythropoiesis. Ann N Y Acad Sci 718:285-299 (1994)); and, like TGFB (Bonewald L F, Mundy G R. Role of Transforming Growth Factor-B in Bone Remodeling. Clin Orthop 250:261-276 (1990)) and bone morphogenetic proteins (BMPs) (Wozney J. The Bone Morphogenetic Protein Family and Osteogenesis. Mol Reprod Dev 32:160-167 (1992)), activin A is abundantly localized in bone matrix (Ogawa Y, Schmidt D K, Nathan R M, Armstrong R M, Miller K L, Sawamura S J, Ziman J M, Erickson K L, de leon E R, Rosen D M. Bovine Bone Activin Enhances Bone Morphogenetic Protein-Induced Ectopic Bone Formation. J Biol Chem 267:14233-37 (1992)). Although inhibin a-subunit expression (required for inhibin dimmer formation) is very low in human and rat bone marrow (Funaba M, Ogawa K, Murata T, Fujimura H, Murata E, Abe M, Takahashi M, Torii K. Follistatin and Activin in Bone: Expression and Localization During Endochondral Bone Development. Endocrinology 137:4250-59 (1996)) (Inoue S, Nomura S, Hosoi T, Ouchi Y, Orimo H, Muramatsu M. Localization ofFollistatin, an Activin-Binding Protein, in Bone Tissues. Calcif Tissue Int 55:395-397 (1994)), inhibin accumulates in the bone marrow, inhibin accumulates in the bone marrow of 25-d-old rats within 10 min of iv injection of [125I]-inhibin A and is retained for at least an hour (Que Y, Kanatani H, Kiyoki M, Eto Y, Ogata E, Matsumoto T. Effect of local Injection of activin A on Bone Formation in Newborn Rats. Bone 15:361-366 (1994)). These results are consistent with the idea that the effects of inhibin on marrow cell hematopoiesis (Yu J, Shao L E, Lemas V, Yu A L, Vaughan J, Rivier J, Vale W. Importance of FSH-Releasing Protein and Inhibin in Erythrodifferentiation. Nature 330:765-767 (1987)) (Fujimoto K, Kawakita M, Kato K, Yonemura Y, Masuda T, Matsuzaki H, Hirose J, Isaji M, Sasaki H, Inoue T. Purification of Megakaryocyte Differentiation Activity from a Human Fibrous Histiocytoma Cell Line: N-Terminal Sequence Homology with Activin A. Biochem. Biophys. Res. Commun. 174:1163-68 (1991)) (Broxmeyer H E, Lu L, Cooper S, Schwall R H, Mason A J, Nikolics K. Selective and Indirect Modulation ofHuman Multipotential and Erythroid Hematopoietic Progenitor Cell Proliferation by Recombinant Human Activin and Inhibin. Proc Natl Acad Sci USA 85:9052-56 (1988)) are attributable to inhibin derived from gonadal sources (Meunier H, Rivier C, Evans R M, Vale W. Gonadal and Extragonadal Expression of S, BA, and BB Subunits in Various Tissues Predicts Diverse Functions. Proc Natl Acad Sci USA 85:247-251 (1988)).

At this time, assays for inhibin A are used to detect ovarian function in assisted reproductive technology and as indicators for ovarian cancer. However, our data suggest a new utility for assaying the Inhibins at other times in which diminished gonadal function is suspected. For example, the serum inhibin A level was a better predictor of bone turnover than bioavailable estradiol or testosterone in our study of women from ages 20-50. Currently, the first endocrine predictor of increased bone turnover that has been associated with the menopause transition is FSH (Ebeling, et al. JCEM 1996: Sep;81(9):3366-71).

Unlike bone mineral density (bone mineral density) measurements, biochemical markers are able to detect acute changes in bone turnover. While bone mineral density tests typically detect bone density changes in years, markers are able to detect changes in bone metabolism in weeks or months. Unlike bone mineral density measurements, however, markers cannot reveal how much bone is present in the skeleton at any given time. For this reason, markers cannot be used to diagnosis osteoporosis or to tell how severe the disease may be.

Two possible indications for biochemical markers are to (1) predict bone loss in peri- and post-menopausal women and to (2) monitor the skeletal response to treatment. After menopause there is an increase in bone turnover, as bone is resorbed faster than it is replaced. This change in bone metabolism results in an increased rate of bone loss, leading to low bone density and increased fracture risk. Women generally lose about one percent of their bone per year during and after menopause. However, a third or more of these women lose bone more rapidly, at a rate of 3 to 5 percent per year. Biochemical markers can help identify these “rapid losers”, individuals who also appear most likely to respond to an osteoporosis therapy. Rapid bone loss can also occur in the elderly and in individuals with diseases co-morbid with osteoporosis, such as hyperparathyroidism and Cushing's syndrome.

Although follow-up bone density measurement is the most accurate means available to monitor the skeletal response, markers may also play a role in evaluating the effects of therapy. Current osteoporosis treatments act to decrease bone resorption, which is detectable by changes in resorption markers. Markers of bone formation can also be used to monitor treatment since inhibition of bone resorption is followed by a coupled decrease in bone formation in individuals receiving therapy.

Studies have found a moderate correlation between decreases in various markers of bone turnover and gains in bone mineral density during treatment. Using markers, the effect of treatment may be determined in a matter of months, while changes in bone density may not be detected for one or two years. Experts suggest that this earlier evidence that an osteoporosis regimen may be working can reinforce a patient's desire to continue therapy, enhancing compliance with treatment. Failure to see a decrease in bone markers could indicate a lack of compliance or efficacy. However, the variability is so great that a second test may be needed to confirm this.

### SUMMARY OF THE INVENTION

The data shown herein is the first data in human subjects to demonstrate that inhibins have suppressive effects on both aspects of bone turnover in vitro (bone formation and bone resorption) through the suppressive effects on differentiation of the cells that contribute to these processes (osteoblasts and osteoclasts). In human subjects, in both in vitro studies (Example 2) and in cross-sectional clinical studies, in women and men (Examples 1 and 3), the data suggest that:

1. The clinical measurement of serum inhibin A levels in pre-menopausal and peri-menopausal women is a useful predictive marker of increased bone turnover, which is an early surrogate for increased bone loss that can be measured 6-12 months after the rate of bone turnover has been elevated.

2. The clinical measurement of serum inhibin B levels in men is a useful marker of decreased spine bone mineral density. The finding that this is a better predictor than steroids suggests that inhibin B levels may be altered in the presence of normal steroid levels, and that inhibin B levels may be a good predictor of bone involutional osteoporosis (decreased gonadal function) and idiopathic osteoporosis (steroid intact gonadal function).

3. Clinical replacement of inhibin A or inhibin B levels may alleviate the increased rate of bone turnover in patients, which would slow the rate of bone loss, and possible prevent osteoporosis or be used as a treatment strategy (through injection or subcutaneous administration) once diagnosis of bone loss has taken place (example 4).

The data shown herein demonstrated that inhibin A was a better predictor of bone turnover than FSH or estradiol in women between ages 20 and 50 (Example 1). Therefore, a clinical determination of decreased serum Inhibin A in women of this age may predict an increase in bone turnover in these women, in the absence of other abnormal hormonal changes.

Additionally, the data shown herein in men with individual bone loss demonstrates that inhibin B is a better predictor of changes in spine bone mineral density than either bioavailable testosterone or bioavailable estradiol, which is currently the state of the art. An assay for inhibin B can be used to diagnose involutional, and idiopathic osteoporosis. BMD correlates with idiopathic or involutional osteoporosis in males. Riggs B L, Khosla S., Melton III LJ 1998. “A unitary model for involutional osteoporosis: estrogen deficiency causes both type I and type II osteoporosis in postmenopausal women and contributes to bone loss in aging men. J Bone Miner Res 13:763-773. Kurland E S, Cosman F., McMahon D J, Rosen C J, Lindsay R., Bilezikian J P. “Parathyroid hormone as a therapy for idiopathic osteoporosis in men: effects on bone mineral density and bone markers.” J Clin Endocrinol Metab. 2000 Sep;85(9):3069-76.

Additionally, this invention provides a method to increase cancellous bone strength in a mammal by administering an effective amount of a derivative of inhibin in a pharmaceutically acceptable carrier to a mammal to increase cancellous bone strength.

Additionally, this invention provides a method to increase bone volume in a mammal by administering an effective amount of a derivative of inhibin in a pharmaceutically acceptable carrier to a mammal to increase bone volume.

### BRIEF DESCRIPTION OF THE DRAWINGS

FIG. 1 shows bone strength (measured by total force) in sham and orchidertomized male mice.

FIG. 2 shows bone volume in sham and orchidertomized male mice.

FIG. 3 shows bone volume in sham and orchidertomized male mice.

FIG. 4 shows total BMD in sham and orchidertomized male mice.

FIG. 5 shows percent change in BMD in sham and orchidertomized male mice.

FIG. 6 shows bone strength (measured by total force) in sham and ovarictomized female mice.

FIG. 7 shows bone volume in sham and ovarictomized female mice.

FIG. 8 shows bone volume in sham and ovarictomized female mice.

### DETAILED DESCRIPTION OF THE INVENTION

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W. H. Freeman and Company, 1993; A. L. Lehninger, Biochemistry (Worth Publishers, Inc., current addition); Sambrook, et al., Molecular Cloning: A Laboratory Manual (2^{nd} Edition, 1989); Methods in Enzymology (S. Colowick and N. Kaplan eds., Academic Press, Inc.); Remington 's Pharmaceutical Sciences, 18^{th} Edition (Easton, Pa.: Mack Publishing Company, 1990); Carey and Sunberg Advanced Organic Chemistry 3^{rd} Ed. (Plenum Press) Vols A and B (1992). All publications, patents and patent applications cited herein, whether supra or infra, are hereby incorporated by reference in their entirety.

In describing the present invention, the following terms will be employed, and are intended to be defined as indicated below.

The term bone turnover refers to the ongoing physiological process of bone formation and bone resorption that occurs to continually replace the skeleton, about once in every 15 years. This normally occurs at a balanced rate, such that bone mass is maintained at a relatively constant level. “Increased bone turnover” usually occurs at a rate that favors more bone resorption than bone formation, since bone resorption takes place in a given site in about three weeks, but requires about three months to refill the same site by bone formation. Thus, measurement of increased bone turnover frequently predicts a future detection of bone loss (measured by bone mineral density).

The term osteopenia refers to the bone density Z score -1.0 standard deviation below the mean bone mineral density of adults of the same age and sex

The term Osteoporosis refers to the bone mineral density S-score 2.5 standard deviation below the mean bone mineral density of adults of the same age and sex.

The term polypeptide refers to a molecule composed of amino acids and the term includes peptides, polypeptides, proteins and peptidomimetics and active polypeptide fragments. The term polypeptide includes chemically modified polypeptides where at least one of its amino acid residues is modified by a natural or chemical modification.

The term small molecule refers to a chemical moiety which may be synthetically produced or obtained from natural sources and typically has a molecular weight of less than 2000 daltons, but more preferably less than 1000 daltons or even less than 600 daltons.

As used herein, the terms “treat” or “treatment” are used interchangeably and are meant to indicate a postponement of development of bone loss symptoms and/or a reduction in the severity of such symptoms that will or are expected to develop. The terms further include ameliorating existing bone or cartilage deficit symptoms, preventing additional symptoms, ameliorating or preventing the underlying metabolic causes of symptoms, and/or encouraging bone growth.

As used herein, the term “subject” encompasses humans either male or female.

Inhibins may be a biomarker more predictive of changes in bone turnover than other currently available assays, such as osteocalcin, estradiol, testosterone, pyridinolines. Inhibin A and B can be used for both diagnosis and for therapeutic uses in individual subjects. The correlation of inhibin A and inhibin B with markers of bone turnover suggest the inhibins regulate bone turnover. More specifically, the detection of inhibin levels can be used to predict bone loss due to increasing bone turnover in male and female subjects. Inhibin A and Inhibin B also have direct suppressive effects on bone marrow cell differentiation in vitro, which is consistent with Inhibins acting to suppress bone turnover through suppression of bone marrow cell differentiation. As a first indication of this function, we have analyzed the inhibin A and inhibin B serum levels in adult women and older men. An inverse correlation for inhibin B was found in women of peri-menopausal age; however, inhibin A levels were inversely correlated with increases in bone formation and bone resorption in both pre-menopausal and peri-menopausal aged women. Inhibin A was shown to be a good predictor of bone turnover in these women. In addition, correlations were also found for inhibin B and bone mineral density (BMD) in older men. Thus, we believe that both analysis of inhibin A and inhibin B levels, and the potential therapeutic manipulation of inhibin levels may have value for the diagnosis and treatment of bone loss due to increasing bone turnover in both women and men.

Inhibin A and inhibin B can be measured in serum by an ELISA assay. The way in which measurement of inhibin A is carried out is not material to the invention. Recently developed specific and sensitive assays for inhibin A are described by Groome et al 1994, Clinical Endocrinology, 40, 717-723; and Muttukrishna et all 994, Human Reproduction 9, 1634-1642. The presently preferred manner for measuring inhibin A in a biological sample uses one antibody specific for the alpha-subunit of inhibin A and a second antibody specific for the beta-subunit of inhibin A. The inhibin A assay has been developed as a 2-site ELISA that selectively measures inhibin A levels. This is available commercially through Diagnostic Systems Laboratory. Normal ranges of Inhibin A in serum is shown in Table 1.

The inhibin B assay can be detected by a 2-site ELISA that selectively measures inhibin B levels through Diagnostic Systems Laboratory. Other flurometric or radioactive assays could be developed by one skilled in the art. In addition to using inhibin assays in the clinic to follow bone turnover, another long term goal is the development of treatment regimens that directly or indirectly increase either inhibins themselves, or the activation of Inhibin signaling on bone marrow cells to suppress their differentiation, and thereby alleviate the increases in bone turnover that are associated with decreases in inhibin levels. Normal ranges of inhibin A and B in serum is shown in Table 1.

TABLE 1

| | | | |
|---|---|---|---|
| For normal pre-menopausal women: | | | |
| Normal ranges of Serum inhibins: | | | |
| Follicular Phase: | Inhibin A | 1-12 | pg/ml |
| | Inhibin B | 100-155 | pg/ml |
| Luteal Phase: | Inhibin A | 3-12 | pg/ml |
| | Inhibin B | 20-70 | pg/ml |
| Peri-menopausal women (>35 yr) in | Inhibin A | −2.1 +/− 0.3 | IU/ml |
| the follicular phase: | Inhibin B | −96 +/ 6 | pg/ml |
| For men: | | | |

| | |
|---|---|
| Normal range of serum inhibin B: | 140-225 pg/ml Inhibin B (Illingworth |
| | et al, JCEM, 1996 V81; 1321-1325) |

The normal range of serum inhibin A is at the limit of detection of the
assay (<15 pg/ml)

Bone turnover is measured by determining the serum levels of bone formation and bone resorption. The bone formation markers used for this determination currently include alkaline phosphatase (AP), bone alkaline phosphatase (BAP), and osteocalcin. The bone resorption markers are all breakdown products of the collagen matrix protein; several assays exist for the measurement of different fragments of the collagen molecule. These include pyridinoline (Pyd), deoxypyridinoline (Dpd), the amino-terminal cross-linked peptide (NTx), and the carboxy-terminal cross-linked peptide (CTx). In addition, urinary products of NTx, CTx, and the N-terminal peptide (N-telopeptide) can also be measured. Combinations of elevated levels outside the clinically defined normal ranges signify increased bone turnover.

Combinations of elevated levels outside the clinically defined normal ranges signify increased bone turnover. These are well established clinically, and the information provided to physicians with the labwork results. Increasing bone turnover involves increases in both bone formation and bone resorption markers, and more resorption than formation leads to bone loss. The normal ranges of bone formation markers are shown in Table 2.

TABLE 2
Normal ranges:

| | |
|---|---|
| Alkaline Phosphatase | 25-165 IU/L |
| Bone Alkaline Phosphatase | 4-35 ng/ml |
| Osteocalcin (OCal) | 3-709 ng/ml |
| Urinary Pyridinium (Pyd) | 20-61 nmol/mmol Creatnine |
| Urinary D-Pyridinium (Dpd) | 4-22 nmol/mmol Creatnine |
| Serum Ntx | 430-570 nmol/ Bone Collagen Equiv/ |
| | mmol Creatnine |
| Serum CTx | <5 ng/ml |
| N-telopeptide | 23-110 nmol Bone Collagen Equiv/ |
| | mmol Creatnine |
| Lumbar spine bone mineral density | >1.150 g/cm2 (T-score by bone |
| | densitometry of <1.0, according to |
| | World Health Organization |
| | guidelines) |
| Total bone mineral density | >1.150 g/cm2 (T-score of <1.0 by |
| | to bone densitometry of <1.0, |
| | according World Health Organization |
| | guidelines) |
| Hip bone mineral density | >1.150 g/cm2 (T-score of <1.0 by |
| | bone densitometry of <1.0, |
| | according to World Health |
| | Organization guidelines) |

In addition to diagnostic tests relating to inhibin, therapeutic treatments are also contemplated because inhibin is a dimeric peptide hormone, it will be a difficult process to generate a small molecule mimetic of the hormone. The most likely possibility is treatment with injectable recombinant human inhibin A, as for insulin and parathyroid hormone (recently manufactured by Lilly Pharmaceutical Forteo™). Injection of recombinant human inhibin A has been used in animal models to regulate reproductive function (for example: Hayes, et al. The Journal of Clinical Endocrinology & Metabolism Vol. 83, No. 6 1835-184; Burger, Hum Reprod. 1993 November;8 Suppl 2:129-32. Review).

To assess the relative contributions of inhibins versus bio-available estradiol in determining bone turnover, multivariate models were constructed in which each marker of bone turnover was the dependent variable, and inhibin A, inhibin B, and bio-available estradiol were the independent variables, after accounting for age. Inhibin B, was not a good predictor of bone formation, or bone resorption when the women were grouped into premenopausal and postmenopausal groups, rather than evaluated in age groups by decade of life. Inhibin B is likely a good predictor of bone turnover in the perimenopausal age group (45-54). However, in pre-menopausal women, inhibin A was a very good predictor of both bone formation and bone resorption, and thus bone turnover. In post-menopausal women inhibin A was the best predictor of bone formation, whereas bio-available estradiol was a better predictor of bone resorption (Example 1).

### EXAMPLE 1

Serum Inhibin A Level is a Better Endocrine Predictor of Increased Bone Turnover than is FSH or Estradiol in Pre-menopausal Women, and Inhibin B is a Good Predictor in Perimenopausal Women.

Human mesenchymal stem cells were grown for 21 days under osteoblastic conditions in the presence or absence of 50 ng/ml inhibin A or inhibin B. Mineralization of the developing osteoblasts was determined by alizarin red staining, normalized to total protein content per well. Both inhibin A and inhibin B suppressed osteoblastogenesis; inhibin B was a more potent suppressor than inhibin A. To determine if serum levels of inhibin B were correlated with markers of bone turnover, a cohort of pre-, peri- and post-menopausal women (n=188, age range 21-85 yrs) were analyzed by decade. Serum and urine samples were collected during the follicular phase of the cycle for pre-menopausal women. Samples were excluded if the women were obtaining estrogen through the use of oral contraceptives or hormone replacement therapy. Consistent with our hypothesis, in the cohort of women of peri-menopausal age (45-54 yrs), but not in any of the other age groups, inhibin B was inversely correlated with Bone Alkaline Phosphatase (BAP; R=0.36, p≦05), with a similar but nonsignificant trend for serum osteocalcin.

Spearman correlation coefficients for serum inhibin A and inhibin B levels were compared with bone formation and resorption markers from women separated into either pre- or post-menopausal groups. When the women were grouped in this way, inhibin A was inversely correlated with all markers of bone turnover measured in pre-menopausal women. These included alkaline phosphatase (AP), bone alkaline phosphatase (BAP), Pyridinoline (Pyd), deoxypyridinoline (Dpd), and the C-terminal peptide cross links of Collagen I (CTx). In addition, the negative correlation of inhibin A with bone formation markers was maintained in post-menopausal women. To assess the relative contributions of inhibins versus bioavailable estradiol in determining bone turnover, multivariate models were constructed in which each marker of bone turnover was the dependent variable, and inhibin A, inhibin B, and bioavailable estradiol were the independent variables after accounting for age. inhibin B, was not a good predictor of bone formation or bone resorption when the women were grouped based upon menopause status, rather than evaluated in age groups by decade of life. However, in pre-menopausal women, inhibin A was a very good predictor of both bone formation and bone resorption, and was a more significant predictor of bone turnover than bioavailable estradiol. In post-menopausal women inhibin A was the best predictor of bone formation, whereas bioavailable estradiol was a better predictor of bone resorption.

To more carefully assess whether changes in inhibin B levels during the menopause transition are correlated with markers of bone turnover, women were separated into age groups by decade. Consistent with our hypothesis and our human in vitro osteoblastogenesis data, inhibin B levels were now inversely correlated with alkaline phosphatase and bone alkaline phosphatase in women selectively during the menopause transition (ages 45-54). Inhibin A levels were inversely correlated with AP, BAP, as well as Dpd and Ctx in young 25-34 year old women not on birth control pills. In addition, like we found for Inhibin B, in women during the menopause transition (ages 45-54), inhibin A levels were also inversely correlated with bone formation markers. These inverse correlations in pre- and peri-menopausal women are consistent with our in vitro data (Example 2) demonstrating that inhibins can suppress osteoblastogenesis. The data in example 1 shows that serum inhibin A levels are useful as predictors of increased bone turnover in both premenopausal and peri-menopausal women. Inhibin B levels are useful as predictors of increased bone turnover only in peri-menopausal women.

The data demonstrated serum concentrations in the following ranges:

TABLE 3

| | | | |
|---|---|---|---|
| Premenopausal women (20-39 yr) | Inhibin A | −20.1 +/− 19.3 | pg/ml |
| in the follicular phase: | Inhibin B | −82.5 + 38.4 | pg/ml |
| Perimenopausal women (40-59 yr) | Inhibin A | −29.6 +/− 30.8 | pg/ml |
| in the follicular phase: | Inhibin B | −62.9 +/− 38.8 | pg/ml |
| All pre-menopausal women in the | Inhibin A | −22.9 +/− 23.9 | pg/ml |
| follicular phase: | Inhibin B | −76.7 +/− 39.1 | pg/ml |
| All post-menopausal women: | Inhibin A | −4.5 +/− 12 | pg/ml |
| | Inhibin B | −12.2 +/− 16.4 | pg/ml |

One limitation is that the assay in women should be performed on blood samples between days 3 and 7 of the menstrual cycle. Thus, if the inhibin A or inhibin B levels are below the normal ranges in the follicular phase it would be more likely that levels of bone turnover markers will be increased. The limitation can be overcome by careful patient monitoring of the cycle, such that serum samples are obtained on the appropriate days 3-7 of the menstrual cycle.

### EXAMPLE 2

Inhibin and Activin Exert Opposing Effects on Osteoblast and Osteoclast Differentiation, and Inhibins Decrease Bone Turnover Through Suppression of Cell Differentiation of Bone Forming Osteoblasts and Bone Resorbing Osteoclasts.

The current study was designed to determine the effects of activin (ActA) and inhibin A (InhA) and inhibin B (InhB) on human osteoblastogenesis and osteoclastogenesis. Inhibin and activin effects on OBL development were assessed using human bone marrow-derived mesenchymal stem cell (MSC) cultures. HMSCs were cultured in osteogenic differentiation medium in the presence or absence of InhA, InhB, InhA+ActA, or InhB+ActA. Osteogenic differentiation was determined on day nine by measuring expression of alkaline phosphatase (AP) and on day 21 by staining mineralized extra cellular matrix. Both InhA and InhB suppressed osteoblastogenesis; the effects of InhB were stronger than that of InhB suppressed osteoblastogenesis; the effects of InhB were stronger than that of InhA. The suppression of OBL development by InhA and InhB was maintained even in the presence of ActA. Surprisingly, ActA stimulated OCL development in human peripheral blood mononuclear cells, even in the presence of excess soluble RANK-Fc, a potent inhibitor of OCL development. These data indicate that human OBL and OCL progenitors are direct targets of inhibin and activin regulation. We hypothesize that changes in the inhibin/activin ratio detected by these cells may alter both OBL and OCL differentiation, thereby contributing to the increased bone resorption observed in perimenopausal women.

### EXAMPLE 3

Inhibin B Is A Good Predictor of Decreased Spine Bone Mineral Density.

We previously demonstrated that inhibins suppress human mesenchymal stem cell osteoblastogenesis, and that decreased serum inhibins in women is associated with increases in markers of bone turnover. To determine if similar correlations exist in men, a cohort of elderly men with FSH levels 1.5 S.D. above the mean (n+76, age range 60-90 yrs) were analyzed. Serum and urine samples were collected, as well as bone mineral density measurements obtained at several sites. In contrast with our previous correlations demonstrated in women, no correlations were found for inhibin B with any serum or urinary markers of bone turnover. However, consistent with our hypothesis, inhibin B was inversely correlated with total body bone mineral density, as well as bone mineral density of the total hip, spine and lateral spine (p<0.01). Multivariate analysis demonstrated that serum Inhibin B was a better predictor of spine and lateral spine bone mineral density than was bioavailable estradiol or testosterone. These novel findings in human samples, along with our previously reported effects of inhibins on both osteoblast and osteoclast development in murine and human cells, indicate that selective changes in inhibin B alter human bone marrow cell differentiation in vitro.

### EXAMPLE 4

Inhibin A can Protect Against Bone Loss and Increase both Bone Mass and Bone Strength in Vivo.

We have previously demonstrated that inhibin-A suppresses and activin-A stimulates osteoblast and osteoclast differentiation in primary Swiss-Webster murine bone marrow cultures, as well as osteoblastogenesis in cultures of primary human bone marrow cells. These data led to our hypothesis that Inhibins act to suppress bone turnover and maintain bone mass through direct inhibitory effects on osteoblast and osteoclast development. To test this hypothesis in vivo in male mice, we utilized a previously published system (Mol Endo, 2000 Jul;14(7):1075-85) which uses transgenic transactivator mice with liver-specific expression of a mifepristone-activated chimeric nuclear receptor (GLVP), crossed with transgenic target mice containing a GVLP-responsive promoter upstream of polio-virus IRES (internal ribosome entry site)-linked sequences coding for the alpha- and beta-subunits of inhibin A. This intercross produced “bigenic” mice capable of regulable expression of inhibin A from the liver, which when induced was associated with suppressed levels of FSH (Mol Endo, 2000 Jul;14(7):1075-85).

We determined that both the GLVP only (monogenic) and the bigenic crossed mouse strains obtained peak bone mass at 5-6 months of age, as determined by bone densitometry using the Piximus (Lunar). At peak bone mass, baseline bone mineral density measurements were performed prior to sham or orchidectomy (ORCH) of male mice and ovariectomy (OVX) of female mice, and the subcutaneous placement of mifepristone or vehicle-containing pellets (Innovative Research). Animals were followed for four weeks prior to obtaining femoral bone marrow for osteogenic culture, and tibial analyses of bone volume by microCT.

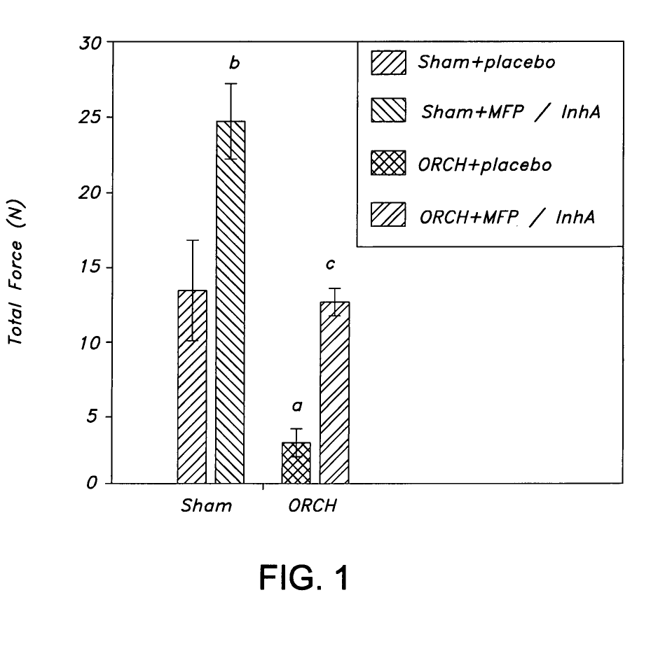

This study showed that over expression of human inhibin A increases cancellous bone strength in intact male mice and maintains bone strength in orchidertomized mice. These results are shown in FIG. 1. The term “sham” refers to intact mice.

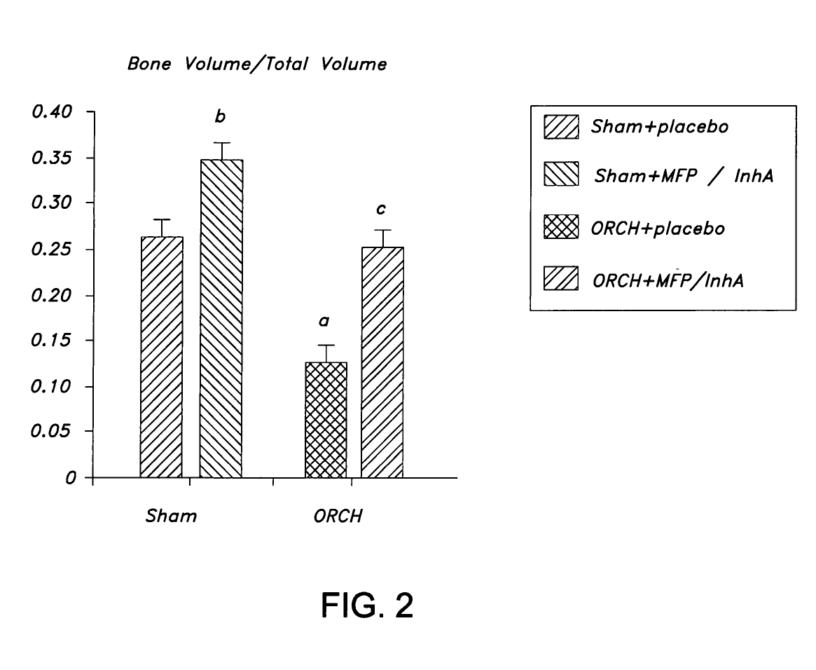

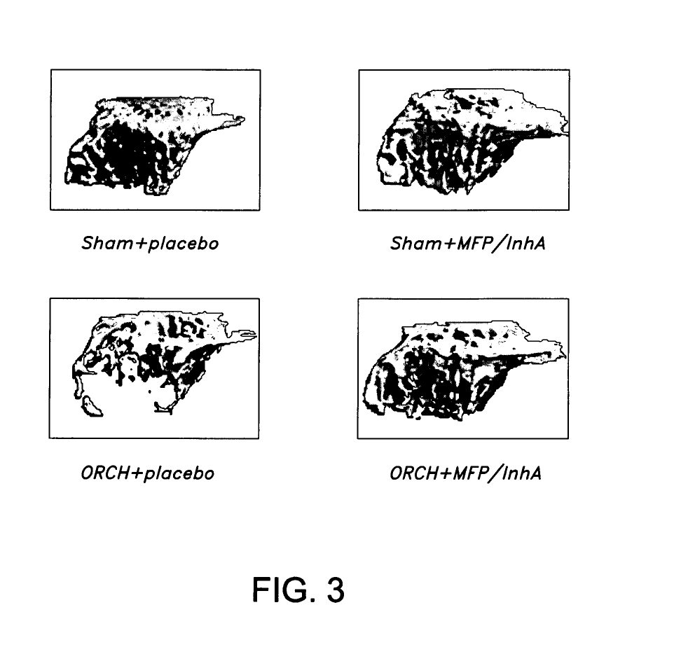

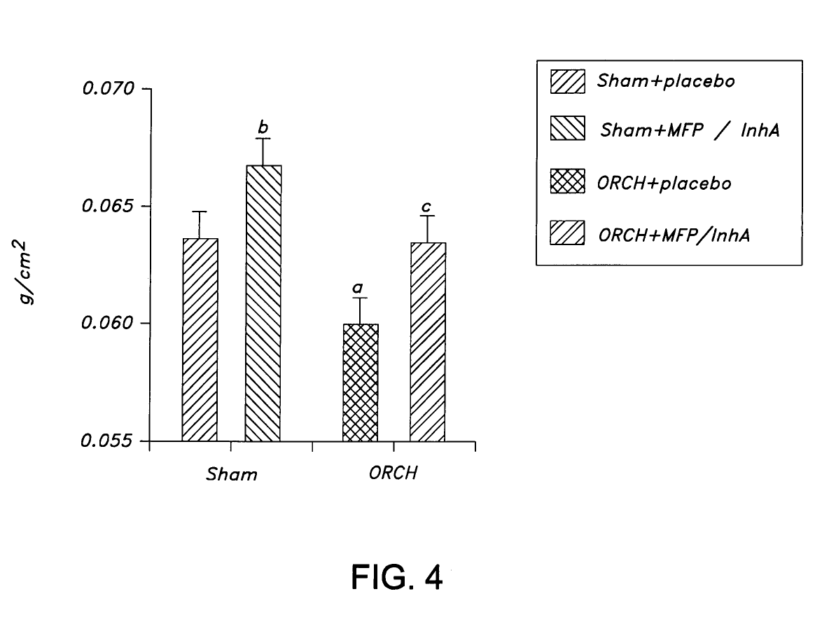

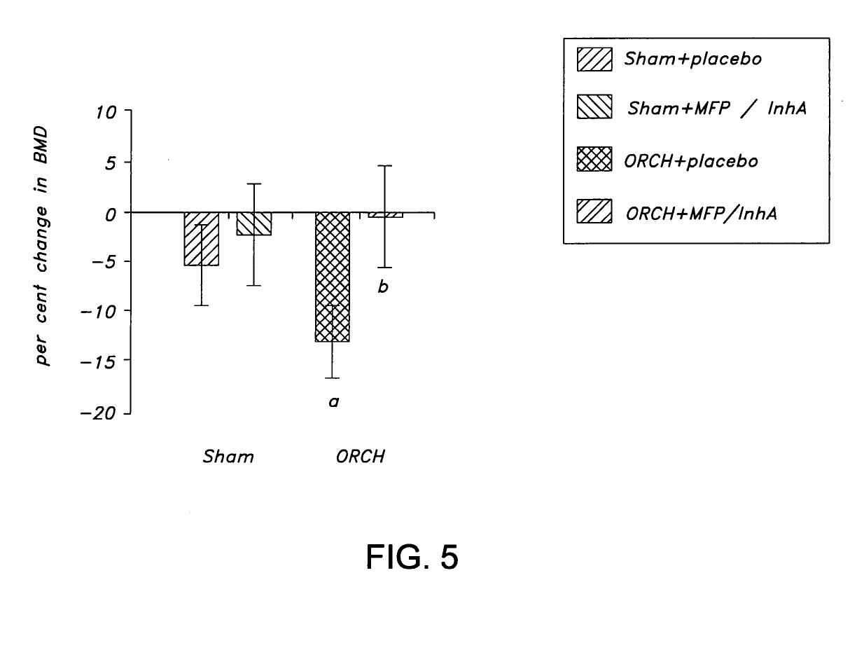

Similarly, as shown in FIGS. 2 and 3, over expression of human inhibin A increases bone volume in intact (sham) male mice and maintains bone volume in orchidectomized male mice. Additionally, over expression of human inhibin A over expression increases total body bone mineral density in male mice as shown in FIG. 4. Similarly, the over expression of inhibin A prevents orchidectomy-induced loss of bone mineral density in male mice as shown in FIG. 5.

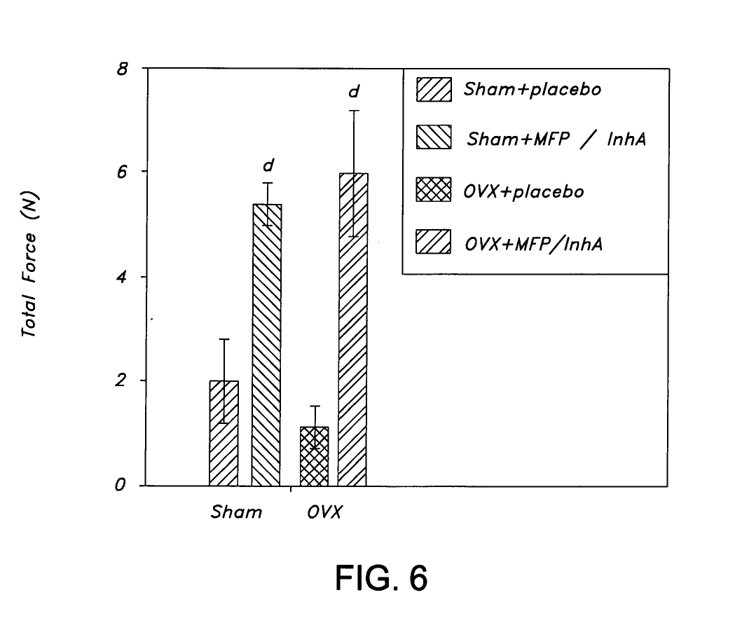

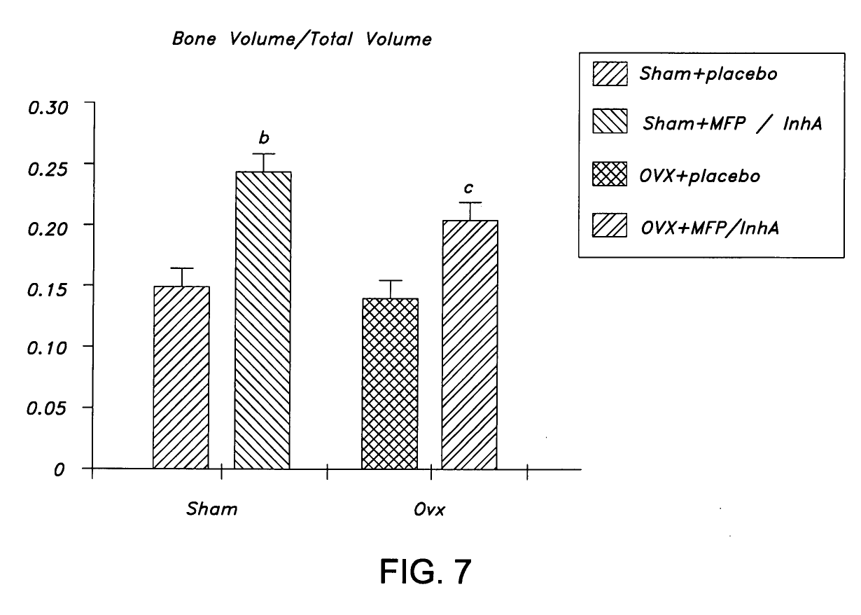

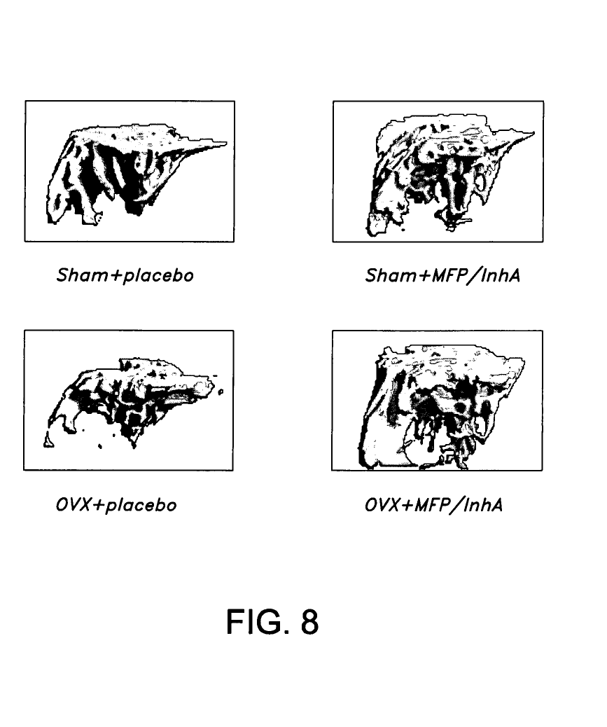

Similarly in female mice, FIG. 6 shows over expression of inhibin A increase cancellous bone strength in intact (sham) and ovarictomized female mice. In FIGS. 7 and 8, over expression of human inhibin A increases bone volume in intact (sham operated) mice and maintains bone volume in ovarictomized (OVX) female mice.

In summary, human inhibin A is a potent anabolic agent that increases bone volume and bone strength. Although these data demonstrate the utility of inhibin A in vivo, replacement with inhibin B may have similar bone protective effects, based upon similar in vitro effects of inhibin A and inhibin B as shown above. Clinical replacement of inhibin A and inhibin B may inhibit the increased rate of bone turnover in patients, which would slow the rate of bone loss; increase bone volume and bone strength. A pharmaceutically acceptable amount of a derivative of inhibin, (or a nontoxic salt thereof) can be combined with a pharmaceutically acceptable carrier to form a pharmaceutical composition. An effective amount of the pharmaceutical composition can be administered through injection or subcutaneous administration to mammals, including humans. Dose response curves to establish an effective amount of the pharmaceutically composition can be determined by one skilled in the art.

A derivative of inhibin is a molecule that is capable of binding inhibin receptors and/or initiating the targeted inhibin—specific cellular responses related to reducing bone turnover, preventing bone loss and/or increasing bone mass. The derivative of inhibin can be protein, peptide or polypeptide recombinantly derived from the cDNA sequence or synthetically produced. Additionally, a derivative of inhibin can be a small molecule agonist. A small molecule agonist can be identified using routine screening methods. Various screening methods can be employed. For example, DOCK3.5, an automatic algorithm to screen small-molecule databes for ligands to fit a given receptor, can be employed. Meng, et al. J. Comp. Chem 15:J05(1992). The identified DOCK3.5 compound can then be used to screen compounds in the available chemical dictionary (Molecular Design Limited, San Leonardo, Calif.) as potential ligands that fit the inhibin receptors. Inhibin binding proteins are known to one skilled in the art. See Structure and Expression of a Membrane Component of the Inhibin Receptor System, 141 Endro. 2600-07 (2000). Vale, et al. Betaglycan as an Inhibin Receptor and uses thereof, U.S. Pat. No. 6,692,744 (Feb. 17, 2004); Daikichi, et al. Novel Polypeptides, cDNA encoding the same and utilization thereof, U.S. patent application Publication No. 20040038285 (Feb. 26, 2004).

The human alpha and beta chains of inhibin and their precursor forms have been isolated and cloned. The sequencing of the inhibin-encoding cDNA has led to the identification of biologically active polypeptides. See Evans, et al., U.S. Pat. No. 4,737,578, showing the cDNA sequence and recombinant cells transformed to express inhibin (hereby specifically incorporated by reference in its entirety). Additionally, recombinant human inhibin A and B can also be generated using stable activin-expressing cell line (PBL, Salk Institute, La Jolla and Diagnostic Systems Laboratories, Webster, Tex.).

Although the foregoing invention has been described in some detail by way of illustration and example for purposes of clarity of understanding, it will be obvious that certain changes and modifications may be practiced within the scope of the appended claims.

I claim:

1. A method to assess bone mineral density in male subject, the method comprising:
a. measuring serum inhibin B level; and
b. correlating said serum inhibin B level with bone mineral density in said male subject.

2. The method of claim 1 wherein said bone mineral density is total bone mineral density.

3. The method of claim 1 wherein said bone mineral density is spine bone mineral density.

4. The method of claim 1 wherein said bone mineral density is hip bone mineral density.

5. The method of claim 1 wherein said serum inhibin B amount is correlated with idiopathic or involutional osteoporosis.

6. A method for diagnosis of increasing bone turnover leading to increased bone loss in premenopausal and postmenopausal women, the method comprising:
a. measuring serum inhibin A level; and
b. correlating said serum inhibin A level with increased bone loss in premenopausal and postmenopausal women.

7. The method of claim 6 further comprising correlating said serum inhibin A levels with a bone turnover marker.

8. The method of claim 7 wherein said bone turnover marker is selected from the group consisting of alkaline phosphatase, deoxypyridinoline, deoxypyridinoline, and C-terminal peptide crosslinks of Collagen I.

9. A method for diagnosis of increasing bone turnover leading to increased bone loss in perimenopausal women, the method comprising:
a. measuring serum inhibin A level; and
b. correlating said serum inhibin A level with increased bone loss in perimenopausal women.

10. The method of claim 9 further comprising correlating said serum inhibin A levels with a bone turnover marker.

11. The method of claim 10 with a bone turnover marker wherein said bone turnover marker is selected from the group consisting of alkaline phosphatase, deoxypyridinoline, deoxypyridinoline, and C-terminal peptide crosslinks of Collagen I.

12. The method to detect increased bone turnover rates in premenopausal women subjects, comprising:
a. measuring serum concentration of inhibin A in said subject;
b. detecting increased bone turnover rates in said subject based on the serum concentration of inhibin A.

13. The method of claim 12 wherein said increased bone turnover rates are predictive of abnormal bone loss.

14. The method of claim 12 wherein said serum is drawn between days 3 to 7 of the subject's menstrual cycle.

15. The method to detect increased bone turnover rates in perimenopausal women subjects, comprising:
a. measuring serum concentration of inhibin B in said subject;
b. detecting increased bone turnover rates in said subject based on the serum concentration of inhibin B.

16. The method of claim 15 wherein said increased bone turnover rates are predictive of abnormal rates of bone loss.

17. The method of claim 15 wherein said serum is drawn between days 3 to 7 of the subject's menstrual cycle.

18. A method to predict bone formation in postmenopausal woman subjects, comprising:
a. measuring serum concentration of inhibin A in said subject; and
b. predicting bone formation in said subject based on the serum concentration of inhibin A.

19. A method to increase cancellous bone strength in a mammal comprising:
a. administering an effective amount of a derivative of inhibin in a pharmaceutically acceptable carrier to a mammal to increase cancellous bone strength.

20. The method of claim 19 wherein said derivative of inhibin is selected from the group consisting of a polypeptide and a small molecule agonist.

21. A method to increase bone volume in a mammal comprising:
a. administering an effective amount of a derivative of inhibin in a pharmaceutically acceptable carrier to a mammal to increase bone volume.

22. The method of claim 21 wherein said derivative of inhibin is selected from a group consisting of a polypeptide and small molecule agonist.
